(19) United States
(12) Patent Application Publication
(10) Pub. No.: US 2011/0224770 A1
(43) Pub. Date: Sep. 15, 2011

(54) Drug Eluting Stents and Methods of Making the Same

(75) Inventors: Pu Zhou, Maple Grove, MN (US); James Feng, Maple Grove, MN (US); Benjamin Arcand, Minneapolis, MN (US); James Lee Shippy, Plymouth, MN (US)

(73) Assignee: BOSTON SCIENTIFIC SCIMED, INC., Maple Grove, MN (US)

(21) Appl. No.: 13/046,970

(22) Filed: Mar. 14, 2011

Related U.S. Application Data
(60) Provisional application No. 61/313,974, filed on Mar. 15, 2010.

(51) Int. Cl. A61F 2/82; B29C 35/02

(52) U.S. Cl. 623/1.2; 623/1.15; 264/232

(57) ABSTRACT

A stent formed from a bioabsorbable polymer matrix including at least one bioabsorbable polymer and at least one therapeutic agent dispersed therein, and methods of making the same.

### CROSS-REFERENCE TO RELATED APPLICATIONS

This application claims the benefit of U.S. Provisional Patent Application No. 61/313,974, filed Mar. 15, 2010, the entire disclosure of which is hereby incorporated herein by reference.

### STATEMENT REGARDING FEDERALLY SPONSORED RESEARCH

Not Applicable.

### BACKGROUND OF THE INVENTION

Various implantable or insertable medical devices have been developed for the delivery of therapeutic agents to the body, many formed from biodegradable or bioresorbable polymer materials, for example, vascular catheter balloons or stents.

In accordance with some delivery strategies, a therapeutic agent is provided within a polymeric coating that is associated with the implantable or insertable medical devices, often metallic devices but polymeric as well. Release of drug from such coatings is generally controlled by simple diffusion. Using coating techniques, however, can result in a significant amount of drug being released prematurely, or even washed from the surface of the device between initial insertion and deployment of the device at the desired lesion site. Premature release can result in imprecision with respect to the amount of drug that is being administered at the lesion site, and also results in drug administration to locations within the body other than the lesion site. Increased drug toxicity can make such events even less desirable.

On the other hand, the sensitivity of therapeutic agents to high temperatures such as those required for extrusion or injection molding of polymer stents, as well as laser cutting, is prohibitive to mixing many therapeutic agents with the polymer material used for stent formation.

Improved control of the drug release profile for implantable and insertable medical devices is desirable.

### SUMMARY OF THE INVENTION

In one aspect, a stent is formed from a bioabsorbable polymer matrix, the bioabsorbable polymer matrix including at least one therapeutic agent dispersed therein.

In another aspect, a stent is formed using a method including forming a paste, the paste including at least one bioabsorbable polymer, at least one therapeutic agent, and at least one solvent, shaping the paste into a stent form and evaporating the solvent.

These and other aspects, embodiments and advantages of the present invention will become immediately apparent to those of ordinary skill in the art upon review of the Detailed Description and Claims to follow.

### BRIEF DESCRIPTION OF THE DRAWINGS

FIG. 1 is illustrates an unexpanded tubular radially expandable stent.

FIG. 2 is a radial cross-section of a stent similar to that shown in FIG. 1 illustrating the therapeutic agent dispersed throughout the polymer matrix forming the stent.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

All US patents and applications and all other published documents mentioned anywhere in this application are incorporated herein by reference in their entirety.

In one aspect, a stent formed from a polymer matrix material includes a therapeutic agent(s) dispersed or distributed within the polymer matrix material.

The stent is suitably formed from bioabsorbable polymer materials, also referred to in the art as biodegradable or bioresorbable polymer materials. Such terms are employed in the art to refer to materials that can be broken down into smaller segments by either chemical or physical process, upon interaction with a physiological environment, for example, erosion or dissolution. These smaller segments can then be eliminated from the physiological environment through metabolization or excretion, for example. Elimination may occur over a period of time from minutes to years, depending on polymer characteristics, such as size and functionality, as well as the physiological environment, while maintaining structural integrity during that same time period.

Bioabsorbable polymers include both naturally occurring polymer materials, as well as synthetic polymer materials.

Examples of classes of bioabsorbable polymer materials include, but are not limited to, polyesters, polyorthoesters, polyanhydrides, polyarylates including L-tyrosine derived and free acid polyarylates, polyanhydrides esters, polyphosphazenes, polycarbonates, particularly the lysine-derived polycarbonates, polyamides (nylon copolyamides), poly(ester-amides), particularly the lysine-containing poly(ester-amides), amino-acid containing polymers including those formed from α-amino acids, polydihydropyrans, polycyanoacrylates, polyketals, polyacetals, poly(imino-carbonates), polyalkylene succinates, polypeptides, polydepsipeptides, etc. These classes of materials include homopolymers, copolymers and terpolymers as well. Blends or mixtures of any of the materials disclosed herein may be employed as well.

Examples of bioabsorbable polyesters include poly(α-hydroxy-esters) and poly(β-hydroxy-esters). Poly(α-hydroxy-esters) include, but are not limited to, polyglycolic acid (PGA), polylactic acid (PLA) and poly(glycolic acid-co-lactic acid) (PLGA). Other suitable polyesters include polycaprolactone, polybutyrolactone and polypropiolactone.

Some exemplary biodegradable polymers for use herein are the a-hydroxy acids polymers including polyglycolic acid (PGA), polylactic acid (PLA), copolymers of glycolic acid and L-, D- or D,L-lactic acid (PLGA), and polycaprolactone, polybutyrolactone, polypropiolactone, poly(hydroxybutyrate-co-valerate), polyanhydrides, polyphosphazenes, polytyrosine derivatives and mixtures thereof. Linear polylactic acid or LPLA is particularly suitable as it is known to undergo significant molecular weight reduction upon exposure to e-beam radiation.

Other exemplary biodegradable polymers are formed from combinations of α-hydroxy acids and α-amino acids, for example, copolymers and terpolymers of lactic acid and/or glycolic acid with serine. See, for example, U.S. Pat. No. 6,042,820, the entire content of which is incorporated by reference herein.

Any of the above biodegradable polymer materials and mixtures thereof may be employed in embodiments herein. The above lists are intended for illustrative purposes only, and not as a limitation on the scope of the present invention.

The polymer matrix material from which the stent is formed may include a therapeutic agent or combination of therapeutic agents dispersed or distributed therein. As employed herein, the term “drug” may be used interchangeably with “active agent”, “therapeutic agent”, “pharmaceutically active agent”, “beneficial agent”, “bioactive agent”, and other related terms may be used interchangeably herein and include genetic therapeutic agents, non-genetic therapeutic agents and cells. A drug may be used singly or in combination with other drugs. Drugs include genetic materials, non-genetic materials, and cells. The term material may be substituted for agent.

In embodiments described herein, drug(s) are intermixed in the polymer matrix that forms the stent structure itself.

Examples of non-genetic therapeutic agents include, but are not limited to, anti-thrombogenic agents, anti-proliferative agents, anti-inflammatory agents, analgesics, antineoplastic/antiproliferative/anti-miotic agents, anesthetic agents, anti-coagulants, vascular cell growth promoters, vascular cell growth inhibitors, cholesterol-lowering agents; vasodilating agents; and agents which interfere with endogenous vascoactive mechanisms.

Genetic agents include anti-sense DNA and RNA and coding DNA, for example.

Cells may be of human origin, animal origin, or may be genetically engineered.

Some exemplary drugs include, but are not limited to, anti-restenosis drugs, such as paclitaxel, sirolimus, everolimus, tacrolimus, dexamethoasone, estradiol, ABT-578 (Abbott Laboratories), trapidil, liprostin, Actinomycin D, Resten-NG, Ap-17, clopidogrel and Ridogrel.

For a more complete discussion of suitable drugs, see, for example, commonly assigned U.S. Publication Nos. 2004/0215169 and 2006/0129727, each of which is incorporated by reference herein in its entirety.

The stents formed herein can be self-expanding or mechanically expandable such as through the use of an expandable balloon. They may be selected from a variety of forms including tubular, sheet, braided, mesh, weave, wire, coil, cut tube, slide-and-lock, etc. Furthermore, the stent can be formed with a variable diameter along its length. One piece bifurcated/branching stents can also be formed herein.

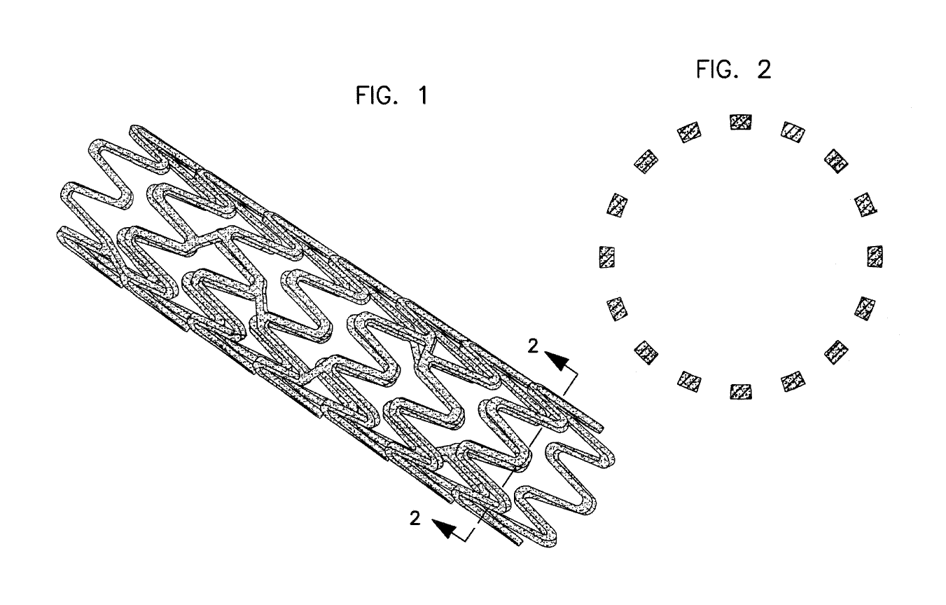

Turning now to the figures, FIG. 1 illustrates generally at 10, one embodiment of a cut tube stent that is a radially expandable stent that is formed herein. In this embodiment, stent 10 includes a plurality of serpentine bands formed of a series of struts 20 and turns 22, each serpentine band interconnected, the stent having therapeutic agent 30 dispersed therein. FIG. 2 is a radial cross-section of a stent 10 similar to that shown in FIG. 1 illustrating struts 20 having therapeutic agent 30 dispersed in the polymer matrix from which struts 20 are formed.

Stent configurations are well known in the art. Any suitable stent configuration may be employed herein. For another type of stent, see for example, U.S. Pat. No. 6,951,053, the entire content of which is incorporated by reference herein. See also U.S. Patent Publication No. 2008/0112999, the entire content of which is incorporated by reference herein.

The therapeutic agent(s) employed herein can be adversely affected upon exposure to high temperature, and it is therefore desirable to employ low temperatures during stent formation. For example, extrusion and injection molding wherein the temperature is greater than that of the polymer melting temperature, as well as laser cutting temperatures can adversely affect therapeutic agent(s). Suitably, the temperature employed for stent formation is less than about 100° F. (about 38° C.). Commonly employed methods of stent formation such as injection molding or extrusion, for example, depend on temperatures of about 40° F. to about 50° F. (or about 20° C. to about 30° C.) higher than the melting point of the polymer material being extruded). This involves temperatures of typically over 300° F. (about 149° C.), and even over 400° F. (about 204° C.). For example, poly(α-hydroxy acids) typically melt in the range of about 325° F. (about 163° C.) to about 450° F. (230° C.) wherein polylactic acid melts at about 325° F. (about 163° C.) and polyglycolic acid melts at 437° F. to 446° F. (about 225° C. to 230° C.).

In one aspect, methods of forming a stent are disclosed wherein the stent is formed from a bioabsorbable polymer matrix material having at least one therapeutic agent dispersed or distributed therein.

In some embodiments, the method includes making a paste, the paste including at least one bioabsorbable polymer material, at least one therapeutic agent and at least one solvent. The paste is then manipulated into a stent configuration and the solvent evaporated.

The paste may suitably include about 5% to about 80% by weight of at least one bioabsorbable polymer, about 0.005 to about 5% by weight of at least one therapeutic agent and about 5% to about 80% by weight of said at least one solvent.

For example, poly(lactic-co-glycolic acid) (PLGA) can be dissolved in dimethylformamide (DMF) up to about 60% by weight. In other instances the polymer may be dissolved up to about 80% by weight. For polymers with higher molecular weight, the paste by be formed with as little as about 5% polymer by weight of the solution.

The percentage by weight of paclitaxel in the final stent (after drying) can range from about 0.005% to 5% by weight depending on stent dimensions and therapeutic agent used. Depending on the therapeutic agent employed, this range may vary. For example, a stent can have higher percentage by weight of everolimus than paclitaxel.

In some embodiments, the stent is formed on a sacrificial mandrel that can be removed after the solvent is evaporated from the paste. The mandrel may be formed of a variety of materials including those that are fluidizable, for example by melting or dissolving, and those that are lubricious wherein the formed stent may be readily removed therefrom.

A specific example of a paste formulation is 45 g of PLGA (50/50 lactide to glycolide), and 0.16 g paclitaxel in 54.84 g DMF.

Mandrels may be formed from materials that can be readily melted at low temperatures, for example, at temperatures or less than that of the polymer from which the stent is formed, and more suitably at temperatures less than those wherein the therapeutic agent may become degraded as discussed above. Examples of materials that may be removed by melting include, but are not limited to, ice, low molecular weight polymers that have a melting point lower than that of the polymer material from which the stent is formed, or a wax such as polyparaffin wax.

In some embodiments, the mandrel is formed from materials other than polymers that readily dissolve, for example, sugar, and dissolvable metals such as magnesium, etc.

In some embodiments, the mandrel is formed from materials that are water soluble or dispersible. Examples of materials are removable by dissolution or water dispersion include, but are not limited to, lubricious hydrogels such as polyvinylpyrrolidone, polyethylene oxide, polyvinyl acetate and polyvinyl alcohol.

Starch (polysaccharide carbohydrate), as well as other natural polysaccharides which are also water dispersible or soluble, can be employed for forming the mandrel.

These fluidizable materials need not be completely melted, dissolved, or otherwise fluidized providing the mandrel has been reduced in size sufficiently to readily release the stent.

In some embodiments, the mandrel is formed from a lubricious material such as a fluoropolymer, for example, polytetrafluoroethylene.

The paste including the polymer, therapeutic agent and solvent may be deposited on the mandrel employing any precise method of pattern forming.

Alternatively, the stent may be formed directly onto a stent delivery device such as a catheter balloon. Because the present method employs such low formation temperatures, no damage is done to the polymer balloon during stent formation because balloons are traditionally formed from polymer materials having significantly higher temperatures than about 100° F. to 150° F. (about 38° C. to 66° C.).

For radially expandable stents that are balloon expandable, the stent is traditionally crimped from a “static state”, i.e. formed state, into a reduced diameter configuration onto the balloon. Forming of the stent directly onto the balloon, eliminates the need for the additional crimping step. Rather, the stent can be formed in the reduced diameter configuration, and then expanded during use.

In one embodiment, the stent is made with a smaller inner diameter than the balloon outer diameter. The stent is then expanded to a larger inner diameter than the balloon outer diameter. The expanded stent is then heat shrunk onto the balloon. The heat shrink temperature is lower than the drug degradation temperature. For PLGA, the shrink temperature is less than 100° C. which is lower than current temperature at which coatings are dried.

In one embodiment, a non-contact direct write MicroPenning® system is employed for stent formation. This system is available from Ohmcraft® Micropen® available from MicroPen® Technologies aka Ohmcraft® located at 93 Paper Mill Street in Honeoye Falls, N.Y. 14472. See http://www.ohmcraft.com/, incorporated by reference herein.

MicroPenning® is a method whereby the polymer matrix material in the form of a paste, is deposited on the mandrel in an additive deposition process, employing consecutive deposition steps until the desired thickness and pattern has been achieved. The method allows for extremely precise deposition of material. Using this method, the mandrel is moved while the dispensing device, which is similar to a syringe or a pen, dispenses the paste onto the mandrel in a predetermined stent pattern controlled via a computer. The paste is pumped from a reservoir to the syringe using micro-capillary technology and dispensed via an extrusion-like mechanism from the syringe onto the mandrel. Features as small as 30-40 μm up to about 150 μm can be achieved with relatively high viscosity liquids.

In another embodiment, a non-contact aerosol jet deposition direct write system is used which is available under the tradename of M³D® from Optomec® at 3911 Singer N.E. Albuquerque, N. Mex. 87109. See http://www.optomec.com/, incorporated by reference herein. This is also a non-contact direct write process that involves aerosolization of conductive pastes which are then formed into a droplet stream of material. Features sizes of less than 20 μm can be obtained, and even as small as 10 μm with low viscosity liquids.

The above described processes can also be employed for providing features to the surface of the device, such as very fine microdots, in addition to making the device itself.

These processes can be used with or without masking. However, in particular embodiments, no masks, screens or stencils are employed.

In some embodiments, these methods are employed to form radial expandable stents. Stents are typically formed in what is referred to in the art as a “static state”. The stent can then be crimped onto a delivery device such as a catheter or balloon, to a reduced diameter configuration. When deployed, the stent is expanded to a diameter size that is larger than that of the static state.

Stent dimensions vary depending on polymer strength, stent configurations and drug concentration. Stent struts can vary in width and thickness from 50 μm to 200 μm. In some embodiments, the struts are 150 μm wide and 150 μm thick.

The polymeric stents disclosed herein are generally formed either in a static state or in a crimped state.

Dispersion of therapeutic agent(s) throughout the polymer matrix from which the stent is formed, allows the stent to continuously elute the agent(s) during their lifetime prior to elimination via dissolution or degradation followed by metabolization, absorption or excretion.

In any of the embodiments disclosed above, the direct write additive deposition methods disclosed herein can be employed to tailor the drug deposition. For example, different drugs having variable doses can be deposited in different locations or in different layers during the stent construction process. This allows for an even more individualized drug release profile.

Drug release profiles vary for individual products. A typical drug release profile will include a strong initial drug burst for about one week, followed by a steady release for the about three months. Using the present method, a stent can be designed to fit this type of drug release profile. For example, with a homogeneous concentration of paclitaxel throughout the stent, the outer layer of the stent can be made of PLGA 50/50. The rest of the stent made of PLGA 75/25. The outer layer will dissolve in the body much faster than rest of stent. Therefore, the drug release will be faster in the initial stage with PLGA 50/50, followed by steady release from the PLGA 75/25 layer.

Of course, the stent can have more than two layers. Each layer can have different thickness. The drug concentration can be different in each layer. The polymer used for each layer can be different as well. The method disclosed herein provides the opportunity to tailor the drug release profile to different situations such as large lumen versus a small lumen, or an average patient versus a diabetic patient.

Forming a stent employing the various methods disclosed herein eliminates many steps that are traditionally used in stent formation such as laser-cutting, cleaning and electropolishing, and depositing a drug-eluting coating over the finished stent surfaces.

In any of the above disclosed embodiments, the stent may further include coatings, protective coatings, etc. These coatings are known to those of ordinary skill in the art. For example, a lubricious coating including a hydrogel, for example polyethylene glycol or polyvinylpyrrolidone, and a crosslinking agent, for example a multifunctional acrylate such as neopentyl glycol diacrylate, can be employed.

Protective coatings may also be polymeric and may include thermoplastic elastomers as well as non-elastomeric polymers. Such coatings may be applied by dissolving the polymer in a solvent and then dipping, brushing or spraying the stent, for example. Protective coatings may also be provided on the surface via plasma polymerization techniques or through the use of hybrid organic-inorganic ceramic materials also known as a sol-gel derived polymer ceramic materials.

The stents disclosed herein can also be provided with radiopaque materials in the form of coatings and markers.

These coatings are intended for illustrative purposes only and not as a limitation on the present invention. Those of ordinary skill in the art understand that there are a variety of lubricious and protective coatings that may be employed herein.

The above examples and disclosure are intended to be illustrative and not exhaustive. These examples and description will suggest many variations and alternatives to one of ordinary skill in this art. All these alternative and variations are intended to be included within the scope of the claims, where the term “comprising” means “including, but not limited to.” Those familiar with the art may recognize other equivalents to the specific embodiments described herein which equivalents are also intended to be encompassed by the claims.

1. A method of forming a bioabsorbable stent, the method comprising:
forming a paste, the paste comprising at least one bioabsorbable polymer, at least one therapeutic agent, and at least one solvent;
shaping said paste into a stent form; and
evaporating the solvent.

2. The method of claim 1 wherein said stent form is a tube, sheet, braid, weave, mesh, wire, coil, cut tube or a slide-and-lock.

3. The method of claim 1 wherein said shaping step is conducted at a temperature of less than about 66° C.

4. The method of claim 1 wherein said shaping step is conducted at a temperature of less than about 38° C.

5. The method of claim 1 wherein said paste comprises about 5% to about 80% by weight of said at least one bioabsorbable polymer, about 0.005 to about 5% by weight of said at least one therapeutic agent and about 5% to about 80% by weight of said at least one solvent.

6. The method of claim 5 wherein said paste comprises about 45% poly(lactide-co-glycolide), about 0.15% paclitaxel and about 54.85% by weight of said at least one solvent.

7. The method of claim 1 further comprising providing a sacrificial mandrel that wherein said stent form is shaped on said mandrel, said mandrel is eliminated after use.

8. The method of claim 1 wherein said stent form is shaped onto an expandable balloon member.

9. The method of claim 1 wherein said shaping step includes forming a pattern with a direct write micropenning system.

10. The method of claim 1 wherein said bioabsorbable polymer matrix comprises at least one member selected from the group consisting of polyglycolic acid, polylactic acid, copolymers of glycolic acid and lactic acid, copolymers and terpolymers of lactic acid, glycolic acid and serine, polycaprolactone, polybutyrolactone, polypropiolactone, poly(hydroxybutyrate-co-valerate), lysine-derived polycarbonate, and mixtures thereof.

11. The method of claim 1 wherein said at least one therapeutic agent is a member selected from the group consisting of paclitaxel, sirolimus, everolimus, tacrolimus, dexamethoasone, estradiol, ABT-578 (Abbott Laboratories), trapidil, liprostin, Actinomycin D, Resten-NG, Ap-17, clopidogrel, Ridogrel and mixtures thereof.

12. A method of forming a bioabsorbable stent, the method comprising:
forming a paste, the paste comprising at least one bioabsorbable polymer, at least one therapeutic agent, and at least one solvent;
shaping said paste into a stent form using a direct write micropenning system; and
evaporating the solvent.

13. The method of claim 12 wherein said stent is formed directly onto a balloon.

14. A stent formed from a bioabsorbable polymer matrix, the bioabsorbable polymer matrix comprising at least one therapeutic agent dispersed therein.

15. The stent of claim 14 wherein said therapeutic agent degrades at temperatures of greater than about 66° C.

16. The stent of claim 14 wherein said therapeutic agent degrades at temperatures of greater than about 38° C.

17. The stent of claim 14 wherein said stent is radial expandable.

18. The stent of claim 17 wherein said stent is self-expanding or balloon expandable.

19. The stent of claim 14 in the form of a tube, sheet, braid, mesh, weave, wire, cut tube, coil, or slide-and-lock.

20. The stent of claim 14 wherein said bioabsorbable polymer matrix comprises at least one member selected from the group consisting of polyesters, polyorthoesters, polyanhydrides, polyarylates, polyanhydrides, polyanhydrides esters, polyphosphazenes, lysine-derived polycarbonates, polyamides, poly(ester-amides), amino-acid containing polymers, polydihydropyrans, polycyanoacrylates, polyketals, polyacetals, poly(imino-carbonates), polyalkylene succinates, polypeptides, polydepsipeptides, polytyrosine derivatives, copolymers and terpolymers thereof, and mixtures thereof.

21. The stent of claim 14 wherein said bioabsorbable polymer matrix comprises at least one member selected from the group consisting of polyglycolic acid, polylactic acid, copolymers of glycolic acid and lactic acid, copolymers and terpolymers of lactic acid, glycolic acid and serine, polycaprolactone, polybutyrolactone, polypropiolactone, poly(hydroxybutyrate-co-valerate), lysine-derived polycarbonate, and mixtures thereof.

22. The stent of claim 14 wherein said bioabsorbable polymer matrix comprises polylactic acid, polyglycolic acid, lysine-derived polycarbonate, copolymers thereof, and mixtures thereof.

23. The stent of claim 14 wherein said at least one therapeutic agent is a member selected from the group consisting of anti-thrombogenic agents, anti-proliferative agents, anti-inflammatory agents, analgesics, antineoplastic/antiproliferative/anti-miotic agents, anesthetic agents, anti-coagulants, vascular cell growth promoters, vascular cell growth inhibitors, cholesterol-lowering agents, vasodilating agents, agents which interfere with endogenous vascoactive mechanisms and mixtures thereof.

24. The stent of claim 14 wherein said at least one therapeutic agent is a member selected from the group consisting of paclitaxel, sirolimus, everolimus, tacrolimus, dexamethoasone, estradiol, ABT-578 (Abbott Laboratories), trapidil, liprostin, Actinomycin D, Resten-NG, Ap-17, clopidogrel, Ridogrel and mixtures thereof.
